(19) United States
(12) Patent Application Publication
(10) Pub. No.: US 2026/0273506 A1
(43) Pub. Date: Sep. 17, 2026

(54) ABSORBENT COMPOSITE MATERIALS, ABSORBENT ARTICLES COMPRISING ABSORBENT COMPOSITE MATERIALS, AND METHODS OF MAKING THEREOF

(71) Applicant: KIMBERLY-CLARK WORLDWIDE, INC., Neenah, WI (US)

(72) Inventors: Xuedong SONG, Neenah, WI (US); Kaiyuan YANG, Neenah, WI (US); Biao FENG, Neenah, WI (US)

(21) Appl. No.: 19/473,227

(22) PCT Filed: May 1, 2024
(86) PCT No.: PCT/US2024/027143

§ 371 Date: Oct. 7, 2025

Related U.S. Application Data
(60) Provisional application No. 63/503,330, filed on May 19, 2023.

(51) Int. Cl. B01J 20/26; A61L 15/60; B01J 20/28; C09K 5/00

(52) U.S. Cl. CPC B01J 20/261; A61L 15/60; B01J 20/28004; B01J 20/28021; B01J 20/28047; C09K 5/00

(57) ABSTRACT

Disclosed herein are absorbent composite materials, absorbent articles comprising said absorbent composite materials, and methods of making thereof. The absorbent composite materials comprising: a superabsorbent material; and a cooling agent; wherein the cooling agent is physically encapsulated within the superabsorbent material. Also disclosed herein are absorbent articles comprising said absorbent composite materials, methods of making the absorbent composite materials, and methods of making the absorbent articles comprising the absorbent composite materials.

### CROSS-REFERENCE TO RELATED APPLICATIONS

This application claims the benefit of priority to U.S. Provisional Application No. 63/503,330 filed May 19, 2023, which is hereby incorporated herein by reference in its entirety.

### BACKGROUND

Absorbent articles such as diapers, training pants, incontinence products, feminine hygiene products, and the like conventionally include a liquid impermeable outer cover and an absorbent core. The absorbent core is typically located between the outer cover and the body of the wearer for taking in and retaining body fluids (e.g., urine) exuded by the wearer.

Thermal comfort has been identified as a priority in delivering overall comfort for absorbent articles. Several strategies have been used to address thermal comfort, including the use of superabsorbent materials. However, more effective materials and methods remain highly desirable. In particular, there is an on-going need in the art for absorbent materials that are suitable for use in absorbent articles and which possess improved processability and thermal management properties. The compositions, products, and methods discussed herein address these and other needs.

### SUMMARY

In accordance with the purposes of the disclosed compositions, products, and methods as embodied and broadly described herein, the disclosed subject matter relates to absorbent composite materials, absorbent articles comprising said absorbent composite materials, and methods of making thereof.

For example, disclosed herein are absorbent composite materials comprising a superabsorbent material and a cooling agent. In various implementations, the cooling agent is physically encapsulated within the superabsorbent material. Also disclosed herein are absorbent articles comprising such absorbent composite materials, methods of making the absorbent composite materials, and methods of making the absorbent articles comprising the absorbent composite materials.

In some examples, the cooling agent is not chemically reacted with the superabsorbent material.

In some examples, the superabsorbent material comprises a plurality of particles, such that the absorbent composite material comprises the cooling agent encapsulated within the plurality of particles. In some examples, the plurality of particles have an average particle size of 20 to 2000 micrometers (microns, μm), such as from 20 to 1000 micrometers, from 60 to 500 micrometers, or from 300 to 600 micrometers.

In some examples, the superabsorbent material comprises a polymer, such as a crosslinked polymer.

In some examples, the superabsorbent material comprises a polymeric hydrogel, such as a crosslinked polymeric hydrogel.

In some examples, the superabsorbent material is derived from acrylic acid, an acrylamide, or a combination thereof.

In some examples, the superabsorbent material comprises a polyacrylic acid-based hydrogel material, an AMPS (acrylamido-2-methylpropane sulfonic acid) based hydrogel material, a polyimine-based hydrogel material, a polyamine-based hydrogel material, a PEG-based hydrogel material, a polysaccharide-based hydrogel material, or a combination thereof.

In some examples, the superabsorbent material is derived from acrylic acid, N,N-methylenebisacrylamide (MBAA), or a combination thereof.

In some examples, the cooling agent has a heat capacity of water of from −20 kJ/kg to −500 kJ/kg.

In some examples, the cooling agent has a molecular weight of from 50 to 1000 Daltons, such as from 50 to 600 Daltons.

In some examples, the cooling agent comprises an anhydrous salt (e.g., a sugar alcohol, urea), a hydrous salt (e.g., a salt hydrate), or a combination thereof.

In some examples, the cooling agent comprises a sugar alcohol, such as xylitol, sorbitol, mannitol, erythritol, or a combination thereof.

In some examples, the cooling agent comprises xylitol, sorbitol, mannitol, erythritol, urea, or a combination thereof.

In some examples, the cooling agent comprises xylitol, urea, or a combination thereof.

In some examples, the absorbent composite material comprises the cooling agent in an amount of from 1% to 50% by weight, based on the total dry weight of the absorbent composite material, such as from 10% to 20%.

In some examples, the absorbent composite material has an absorbency under load and the absorbency under load of the absorbent composite material is improved (e.g., increased) relative to that of the superabsorbent material in the absence of the encapsulated cooling agent. In some examples, the absorbency under load of the absorbent composite material is increased by an amount of from 5% to 40%, such as from 10% to 25%, relative to that of the superabsorbent material in the absence of the encapsulated cooling agent.

In some examples, the absorbent composite material has a centrifuge retention capacity and the centrifuge retention capacity of the absorbent composite material is improved (e.g., increased) relative to that the superabsorbent material in the absence of the encapsulated cooling agent. In some examples, the centrifuge retention capacity of the absorbent composite material is improved (e.g., increased) by an amount of from 5% to 40%, such as from 10 to 25%, relative to that of the superabsorbent material in the absence of the encapsulated cooling agent.

In some examples, upon liquid insult with a liquid at 35° C., the absorbent composite material exhibits an increase in temperature and the increase in temperature of the absorbent composite material is less than the increase in temperature of the superabsorbent material in the absence of the encapsulated cooling agent. In some examples, the increase in temperature of the absorbent composite material is less than the increase in temperature of the superabsorbent material in the absence of the encapsulated cooling agent by an amount of from 0.1° C. to 5° C., such as from 0.4° C. to 2° C., or from 0.4° C. to 1° C.

In some examples, when the absorbent composite material is subjected to liquid insult with a liquid at 35° C. and subsequently left at a temperature of 35° C. and 70% relative humidity or more for 8 hours, then the absorbent composite material releases moisture vapor in an amount, and the amount of vapor released from the absorbent composite material is less than that of the superabsorbent material in the absence of the encapsulated cooling agent. In some examples, the amount of moisture vapor released from the absorbent composite material is less than that of the superabsorbent material in the absence of the encapsulated cooling agent by an amount of from 1% to 40%, such as from 10% to 30%.

In some examples, the absorbent composite material provides improved thermal comfort, absorbency, antimicrobial control, odor control, skin health, or a combination thereof relative to the superabsorbent material in the absence of the encapsulated cooling agent.

Also disclosed herein are absorbent articles comprising any of the absorbent composite materials disclosed herein.

In some examples, the absorbent article is configured to be worn by a person. In some examples, the absorbent article is a diaper, toilet training pant, incontinence product, or feminine hygiene product.

In some examples, the absorbent article is configured to be subjected to a liquid insult. In some examples, the liquid insult comprises contact with urine, menses, perspiration, or a combination thereof.

In some examples, the absorbent article comprises an outer cover and an absorbent core comprising the absorbent composite material. In some examples, the outer cover has an interior surface and an exterior surface, and the absorbent core has a first surface positioned adjacent to the interior surface of the outer cover and a second surface that faces a wearer, the second surface being opposite of the first surface. In some examples, the absorbent core is airlaid. In some examples, the absorbent core further comprises one or more additional materials, such as cellulose fibers, an adhesive, or a combination thereof.

Also disclosed herein are methods of making any of the absorbent articles disclosed herein. For example, the methods can comprise depositing the absorbent composite material to form the absorbent article. In some examples, the method comprises air laying the absorbent composite material. In some examples, the absorbent composite material exhibits improved processability relative to the superabsorbent material and the cooling agent separately. In some examples, the method further comprises making the absorbent composite material.

Also disclosed herein are methods of making any of the absorbent composite materials disclosed herein.

The details of one or more implementations of the present disclosure are set forth in the accompanying drawings and the description below. Other features, objects, and advantages of the present disclosure will be apparent from the description and drawings, and from the claims. Additional advantages of the disclosed compositions, products, and methods will be set forth in part in the description which follows, and in part will be obvious from the description. The advantages of the disclosed compositions, products, and methods will be realized and attained by means of the elements and combinations particularly pointed out in the appended claims. It is to be understood that both the foregoing general description and the following detailed description are exemplary and explanatory only and are not restrictive of the disclosed systems and methods, as claimed.

### BRIEF DESCRIPTION OF THE FIGURES

The accompanying figures, which are incorporated in and constitute a part of this specification, illustrate several aspects of the disclosure, and together with the description, serve to explain the principles of the disclosure.

FIG. 1 is a graph showing a dynamic vapor release profile of superabsorbent materials with and without urea (35° C. and 70% relative humidity).

FIG. 2 is a graph showing a dynamic vapor release profile of superabsorbent materials with and without urea (25° C. and 95% relative humidity).

FIG. 3 is a graph showing a dynamic vapor release profile of superabsorbent materials with and without xylitol (25° C. and 70% relative humidity).

FIG. 4 is a graph showing shear storage modulus as a function of xylitol loading.

### DETAILED DESCRIPTION

The compositions, products, and methods described herein may be understood more readily by reference to the following detailed description of specific aspects of the disclosed subject matter and the Examples included therein. Before the present compositions, products, and methods are disclosed and described, it is to be understood that the aspects described below are not limited to specific synthetic methods or specific reagents, as such may, of course, vary. It is also to be understood that the terminology used herein is for the purpose of describing particular aspects only and is not intended to be limiting.

Also, throughout this specification, various publications are referenced. The disclosures of these publications in their entireties are hereby incorporated by reference into this application in order to more fully describe the state of the art to which the disclosed matter pertains. The references disclosed are also individually and specifically incorporated by reference herein for the material contained in them that is discussed in the sentence in which the reference is relied upon.

### General Definitions

In this specification and in the claims that follow, reference will be made to a number of terms, which shall be defined to have the following meanings.

Throughout the description and claims of this specification, the word “comprise” and other forms of the word, such as “comprising” and “comprises,” means including but not limited to, and is not intended to exclude, for example, other additives, components, integers, or steps.

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Values can be expressed herein as an “average” value. “Average” generally refers to the statistical mean value.

By “substantially” is meant within 5%, e.g., within 4%, 3%, 2%, or 1%.

“Exemplary” means “an example of” and is not intended to convey an indication of a preferred or ideal implementation. “Such as” is not used in a restrictive sense, but for explanatory purposes.

It is understood that throughout this specification the identifiers “first” and “second” are used solely to aid in distinguishing the various components and steps of the disclosed subject matter. The identifiers “first” and “second” are not intended to imply any particular order, amount, preference, or importance to the components or steps modified by these terms.

References in the specification and concluding claims to parts by weight of a particular element or component in a composition denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

A weight percent (wt. %) of a component, unless specifically stated to the contrary, is based on the total weight of the formulation or composition in which the component is included.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, by a “subject” is meant an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

“Biocompatible” and “biologically compatible”, as used herein, generally refer to compounds and/or compositions that are, along with any metabolites or degradation products thereof, generally non-toxic to normal cells and tissues, and which do not cause any significant adverse effects to normal cells and tissues when cells and tissues are incubated (e.g., cultured) in their presence.

As used herein, “antimicrobial” refers to the ability to treat or control (e.g., reduce, prevent, treat, or eliminate) the growth of a microbe at any concentration. Similarly, the terms “antibacterial,” “antifungal,” and “antiviral” refer to the ability to treat or control the growth of bacteria, fungi, and viruses at any concentration, respectively.

The term “(co) polymer” includes homopolymers, copolymers, or mixtures thereof.

The term “(meth)acryl . . . ” includes “acryl . . . ,” “methacryl . . . ,” or mixtures thereof.

As used herein, “molecular weight” refers to the number average molecular weight as measured by ¹H NMR spectroscopy, unless indicated otherwise.

As used herein, the term “absorbent article” generally refers to an article which can absorb and contain various liquids, solids, and semi-solids. For example, personal care absorbent articles refer to devices or products which are placed against or near the skin to absorb and contain the various liquid, solid, and semi-solid exudates discharged from the body. In some examples, the absorbent articles can be disposable.

The term “disposable” is used herein to describe absorbent articles that are not intended to be laundered or otherwise restored or reused as an absorbent article after a single use.

Absorbent articles include, but not are not limited to, diapers, diaper pants, training pants, youth pants, swim pants, feminine hygiene products (including, but not limited to, menstrual pads or pants), incontinence products and other adult care garments, medical garments, surgical pads and bandages, other personal care or health care garments.

The term “health/medical absorbent articles” includes a variety of professional and consumer health-care products including, but not limited to, products for applying hot or cold therapy, medical gowns (e.g., protective and/or surgical gowns), surgical drapes, caps, gloves, face masks, bandages, wound dressing, wipes, covers, containers, filters, medical absorbent garments, disposable garments and bed pads, under pads, and the like.

The term “household/industrial absorbent articles” includes construction and packaging Supplies, products for cleaning and disinfecting, wipes, covers, filters, towels, dis posable cutting sheets, bath tissue, facial tissue, nonwoven roll goods, home-comfort products including pillows, pads, mats, cushions, masks and body care products such as products used to cleanse or treat the skin, laboratory coats, coveralls, trash bags, pet care absorbent liners, laundry soil/ink absorbers, and the like.

The term “personal care absorbent articles” includes, but is not limited to, absorbent articles such as diapers, diaper pants, baby wipes, training pants, absorbent underpants, child care pants, Swimwear, and other disposable garments; feminine care products including sanitary napkins, wipes, menstrual pads, menstrual pants, panty liners, panty shields, interlabials, tampons, and tampon applicators; adult-care products including wipes, pads such as breast pads, containers, incontinence products, and urinary shields; clothing components; bibs; athletic and recreation products; and the like.

The term “sports/construction absorbent articles” includes headbands, wrist bands and other aids for absorption of perspiration, absorptive windings for grips and handles of sports equipment, and towels or absorbent wipes for cleaning and drying off equipment during use.

The terms “nonwoven” and “nonwoven web” refer to materials and webs of material having a structure of individual fibers or filaments which are interlaid, but not in an identifiable manner as in a knitted fabric. The terms “fiber” and “filament” are used herein interchangeably. Nonwoven fabrics or webs have been formed from many processes such as, for example, meltblown processes, spunbond processes, air laying processes, wet laying processes and bonded carded-web processes.

### Absorbent Composite Materials

Disclosed herein are absorbent composite materials comprising a cooling agent physically encapsulated within a superabsorbent material. In various implementations, the cooling agent is physically encapsulated within the superabsorbent material such that the cooling agent is not chemically reacted with the superabsorbent material. As used herein, the term “physically encapsulated” means that the cooling agent is trapped within the superabsorbent material and is not chemically reacted with the superabsorbent material. Despite being encapsulated by the superabsorbent material, the cooling agent surprisingly imparts excellent thermal management properties to the absorbent composite material. The cooling agent was also surprisingly discovered to contribute positively to absorbency, such as absorbency under load and centrifugal retention capacity. Furthermore, the absorbent composite material has excellent processability characteristics due, at least in part, to the encapsulation of the cooling agent within the superabsorbent material. As a result, the absorbent composite material provides a combination of excellent processability, thermal management, and absorptive properties, rendering it highly effective for use within absorbent articles (e.g., diapers, training pants, incontinence products, feminine hygiene products, and the like).

### Superabsorbent Materials

Superabsorbent materials are well known in the art and can be selected from natural, synthetic, and modified natural polymers and materials. For example, superabsorbent materials used to encapsulate a cooling agent in the absorbent composite material can be inorganic materials, such as silica gels, or organic compounds, such as crosslinked polymers. In some examples, superabsorbent materials used to encapsulate a cooling agent in the absorbent composite material can be organic compounds, such as crosslinked polymers.

Typically, a superabsorbent material is capable of absorbing from 10 to 1000 times its weight in liquid, such as an aqueous solution containing about 0.9 weight percent sodium chloride. For example, the superabsorbent material can absorb 10 times or more its weight in liquid, such as an aqueous solution containing about 0.9 weight percent sodium chloride (e.g., 15 times or more, 20 times or more, 25 times or more, 30 times or more, 35 times or more, 40 times or more, 45 times or more, 50 times or more, 60 times or more, 70 times or more, 80 times or more, 90 times or more, 100 times or more, 125 times or more, 150 times or more, 175 times or more, 200 times or more, 225 times or more, 250 times or more, 300 times or more, 350 times or more, 400 times or more, 450 times or more, 500 times or more, 550 times or more, 600 times or more, 650 times or more, 700 times or more, 750 times or more, 800 times or more, 850 times or more, or 900 times or more). In some examples, the superabsorbent material can absorb 1000 times or less its weight in liquid, such as an aqueous solution containing about 0.9 weight percent sodium chloride (e.g., 950 times or less, 900 times or less, 850 times or less, 800 times or less, 750 times or less, 700 times or less, 650 times or less, 600 times or less, 550 times or less, 500 times or less, 450 times or less, 400 times or less, 350 times or less, 300 times or less, 250 times or less, 225 times or less, 200 times or less, 175 times or less, 150 times or less, 125 times or less, 100 times or less, 90 times or less, 80 times or less, 70 times or less, 60 times or less, 50 times or less, 45 times or less, 40 times or less, 35 times or less, 30 times or less, 25 times or less, or 20 times or less). The amount of liquid that the superabsorbent material can absorb can range from any of the minimum values described above to any of the maximum values described above. For example, the superabsorbent material can absorb from 10 to 1000 times its weight in liquid, such as an aqueous solution containing about 0.9 weight percent sodium chloride (e.g., from 10 to 500 times its weight in liquid, from 500 to 1000 times its weight in liquid, from 10 to 200 times its weight in liquid, from 200 to 400 times its weight in liquid, from 400 to 600 times its weight in liquid, from 600 to 800 times its weight in liquid, from 800 to 1000 times its weight in liquid, from 10 to 800 times its weight in liquid, from 10 to 600 times its weight in liquid, from 10 to 400 times its weight in liquid, from 10 to 100 times its weight in liquid, from 50 to 1000 times its weight in liquid, from 100 to 1000 times its weight in liquid, from 200 to 1000 times its weight in liquid, from 400 to 1000 times its weight in liquid, from 600 to 1000 times its weight in liquid, from 900 to 1000 times its weight in liquid, from 20 to 950 times its weight in liquid, or from 50 to 900 times its weight in liquid).

The superabsorbent material encapsulating the cooling agent in the absorbent composite material can, for example, comprise a polymer, such as a crosslinked polymer. In some examples, the superabsorbent material comprises a polymeric hydrogel, such as a crosslinked polymeric hydrogel. Examples of crosslinked polymeric hydrogels include, but are not limited to, neutral and charged polymeric hydrogel materials. Examples of charged polymeric hydrogels include, for example, negatively charged, positively charged, and zwitterionic polymers. Examples of negatively charged polymeric hydrogels include, but are not limited to, polyacrylic acid-based materials, AMPS (acrylamido-2-methylpropane sulfonic acid) based hydrogel materials, and hydrogels of copolymers of AA-AMPS. Examples of positively charged hydrogel materials include, but are not limited to, polyimine and polyamine. Examples of crosslinked neutral polymeric hydrogels include, but are not limited to, PEG-based and polysaccharide-based materials. Other examples of highly absorbent polymers include, but are not limited to, starch-based, cellulosic-based and synthetic polymer-based polymers, such as starch-acrylic acid graft copolymers and starch-acrylonitrile copolymers. Additional examples of superabsorbent materials include those obtained by partially cross-linking a water-soluble polymer such as starch, polyvinyl alcohol, carboxymethyl cellulose, and polyacrylic acid with sodium acrylate.

In some examples, the superabsorbent material comprises a polyacrylic acid-based hydrogel material, an AMPS (acrylamido-2-methylpropane sulfonic acid) based hydrogel material, a polyimine-based hydrogel material, a polyamine-based hydrogel material, a PEG-based hydrogel material, a polysaccharide-based hydrogel material, or a combination thereof.

In some examples, the superabsorbent material is derived from acrylic acid, an acrylamide, or a combination thereof. In some examples, the superabsorbent material is derived from acrylic acid, N,N-methylenebisacrylamide (MBAA), or a combination thereof.

The superabsorbent material can be in any suitable form, such as particles, fibers, sheets, etc. In some examples, the superabsorbent material comprises a plurality of particles, such that the absorbent composite material comprises the cooling agent encapsulated within the plurality of particles. The plurality of particles can be of any shape (e.g., a sphere, a rod, a quadrilateral, an ellipse, a triangle, a polygon, etc.). In some examples, the plurality of particles can have a regular shape, an irregular shape, an isotropic shape, an anisotropic shape, or a combination thereof. In some examples, the plurality of particles are substantially spherical in shape.

The plurality of particles can have an average particle size. “Average particle size” and “mean particle size” are used interchangeably herein, and generally refer to the statistical mean particle size of the particles in a population of particles. For example, the average particle size for a plurality of particles with a substantially spherical shape can comprise the average diameter of the plurality of particles. For a particle with a substantially spherical shape, the diameter of a particle can refer, for example, to the physical diameter. As used herein, the physical diameter of a particle can refer to the largest linear distance between two points on the surface of the particle. Mean particle size can be measured using methods known in the art, such as evaluation by scanning electron microscopy, transmission electron microscopy, and/or optical microscopy.

In some examples, the plurality of particles can have an average particle size of 20 micrometers (microns, μm) or more (e.g., 25 μm or more, 30 μm or more, 35 μm or more, 40 μm or more, 45 μm or more, 50 μm or more, 60 μm or more, 70 μm or more, 80 μm or more, 90 μm or more, 100 μm or more, 125 μm or more, 150 μm or more, 175 μm or more, 200 μm or more, 225 μm or more, 250 μm or more, 300 μm or more, 350 μm or more, 400 μm or more, 450 μm or more, 500 μm or more, 550 μm or more, 600 μm or more, 650 μm or more, 700 μm or more, 750 μm or more, 800 μm or more, 850 μm or more, 900 μm or more, 950 μm or more, 1000 μm or more, 1100 μm or more, 1200 μm or more, 1300 μm or more, 1400 μm or more, 1500 μm or more, 1600 μm or more, 1700 μm or more, or 1800 μm or more). In some examples, the plurality of particles can have an average particle size of 2000 μm or less (e.g., 1900 μm or less, 1800 μm or less, 1700 μm or less, 1600 μm or less, 1500 μm or less, 1400 μm or less, 1300 μm or less, 1200 μm or less, 1100 μm or less, 1000 μm or less, 950 μm or less, 900 μm or less, 850 μm or less, 800 μm or less, 750 μm or less, 700 μm or less, 650 μm or less, 600 μm or less, 550 μm or less, 500 μm or less, 450 μm or less, 400 μm or less, 350 μm or less, 300 μm or less, 250 μm or less, 225 μm or less, 200 μm or less, 175 μm or less, 150 μm or less, 125 μm or less, 100 μm or less, 90 μm or less, 80 μm or less, 70 μm or less, 60 μm or less, 50 μm or less, 45 μm or less, 40 μm or less, 35 μm or less, or 30 μm or less). The average particle size of the plurality of particles can range from any of the minimum values described above to any of the maximum values described above. For example, the plurality of particles can have an average particle size of from 20 to 2000 micrometers (microns, μm) (e.g., from 20 to 1000 μm, from 1000 to 2000 μm, from 20 to 500 μm, from 500 to 1000 μm, from 1000 to 1500 μm, from 1500 to 2000 μm, from 20 to 1750 μm, from 20 to 1500 μm, from 20 to 750 μm, from 20 to 250 μm, from 50 to 2000 μm, from 100 to 2000 μm, from 250 to 2000 μm, from 500 to 2000 μm, from 750 to 2000 μm, from 1750 to 2000 μm, from 25 to 1900 μm, from 30 to 1800 μm, from 40 to 1500 μm, from 50 to 1000 μm, from 60 to 500 μm, or from 300 to 600 μm). In some examples, the plurality of particles can have an average particle size of from 20 to 1000 micrometers, from 60 to 500 micrometers, or from 300 to 600 micrometers.

### Cooling Agents

The cooling agent used in the absorbent composite material (and encapsulated by the superabsorbent material) can comprise any suitable material. In various implementations, the cooling agent can be a material that provides a cooling effect or sensation when contacted with a liquid insult or aqueous solution. For example, the cooling agent can be a material that absorbs heat via heat of dissolution, heat of hydration, heat of reaction, or the like.

In some examples, the cooling agent can comprise a hydrous salt (e.g., a salt hydrate), an anhydrous salt, or a combination thereof. Examples of hydrous salts include, but are not limited to, such as sodium acetate, sodium carbonate, sodium sulfate, sodium thiosulfate, and sodium phosphate, and anhydrous salts such as ammonium nitrate, potassium nitrate, ammonium chloride, potassium chloride, and sodium nitrate. Examples of anhydrous salts include, but are not limited to urea, ammonium nitrate, potassium nitrate, ammonium chloride, potassium chloride, sodium nitrate, sugar alcohols, and the like. Examples of sugar alcohols include, but are not limited to, xylitol, sorbitol, mannitol, erythritol, and combinations thereof.

In some examples, the cooling agent comprises xylitol, sorbitol, mannitol, erythritol, urea, or a combination thereof. In some examples, the cooling agent comprises xylitol, urea, or a combination thereof.

In various implementations, the cooling agent can have a heat capacity of water of −20 kJ/kg or less (e.g., −25 kJ/kg or less, −30 kJ/kg or less, −35 kJ/kg or less, −40 kJ/kg or less, −45 kJ/kg or less, −50 kJ/kg or less, −60 kJ/kg or less, −70 kJ/kg or less, −80 kJ/kg or less, −90 kJ/kg or less, −100 kJ/kg or less, −125 kJ/kg or less, −150 kJ/kg or less, −175 kJ/kg or less, −200 kJ/kg or less, −225 kJ/kg or less, −250 kJ/kg or less, −275 kJ/kg or less, −300 kJ/kg or less, −325 kJ/kg or less, −350 kJ/kg or less, −375 kJ/kg or less, −400 kJ/kg or less, −425 kJ/kg or less, or −450 kJ/kg or less). In some examples, the cooling agent can have a heat capacity of water of −500 kJ/kg or more (e.g., −475 kJ/kg or more, −450 kJ/kg or more, −425 kJ/kg or more, −400 kJ/kg or more, −375 kJ/kg or more, −350 kJ/kg or more, −325 kJ/kg or more, −300 kJ/kg or more, −275 kJ/kg or more, −250 kJ/kg or more, −225 kJ/kg or more, −200 kJ/kg or more, −175 kJ/kg or more, −150 kJ/kg or more, −125 kJ/kg or more, −100 kJ/kg or more, −90 kJ/kg or more, −80 kJ/kg or more, −70 kJ/kg or more, −60 kJ/kg or more, −50 kJ/kg or more, −45 kJ/kg or more, −40 kJ/kg or more, −35 kJ/kg or more, or −30 kJ/kg or more). The heat capacity of water of the cooling agent can range from any of the minimum values described above to any of the maximum values described above. For example, the cooling agent can have a heat capacity of water of from −20 kJ/kg to −500 kJ/kg (e.g., from −20 to −250 kJ/kg, from −250 to −500 kJ/kg, from −20 to −100 kJ/kg, from −100 to −200 kJ/kg, from −200 to −300 kJ/kg, from −300 to −400 kJ/kg, from −400 to −500 kJ/kg, from −20 to −400 kJ/kg, from −20 to −300 kJ/kg, from −20 to −200 kJ/kg, from −50 to −500 kJ/kg, from −100 to −500 kJ/kg, from −200 to −500 kJ/kg, from −300 to −500 kJ/kg, from −25 to −450 kJ/kg, from −50 to −400 kJ/kg, from −75 to −350 kJ/kg, from −100 to −300 kJ/kg, or from −125 to −250 kJ/kg).

In some examples, the cooling agent has a molecular weight of 50 Daltons or more (e.g., 60 Daltons or more, 70 Daltons or more, 80 Daltons or more, 90 Daltons or more, 100 Daltons or more, 125 Daltons or more, 150 Daltons or more, 175 Daltons or more, 200 Daltons or more, 225 Daltons or more, 250 Daltons or more, 300 Daltons or more, 350 Daltons or more, 400 Daltons or more, 450 Daltons or more, 500 Daltons or more, 550 Daltons or more, 600 Daltons or more, 650 Daltons or more, 700 Daltons or more, 750 Daltons or more, 800 Daltons or more, 850 Daltons or more, or 900 Daltons or more). In some examples, the cooling agent has a molecular weight of 1000 Daltons or less (e.g., 950 Daltons or less, 900 Daltons or less, 850 Daltons or less, 800 Daltons or less, 750 Daltons or less, 700 Daltons or less, 650 Daltons or less, 600 Daltons or less, 550 Daltons or less, 500 Daltons or less, 450 Daltons or less, 400 Daltons or less, 350 Daltons or less, 300 Daltons or less, 250 Daltons or less, 225 Daltons or less, 200 Daltons or less, 175 Daltons or less, 150 Daltons or less, 125 Daltons or less, 100 Daltons or less, 90 Daltons or less, 80 Daltons or less, or 70 Daltons or less). The molecular weight of the cooling agent can range from any of the minimum values described above to any of the maximum values described above. For example, the cooling agent can have a molecular weight of from 50 to 1000 Daltons (e.g., from 50 to 500 Daltons, from 500 to 1000 Daltons, from 50 to 200 Daltons, from 200 to 400 Daltons, from 400 to 600 Daltons, from 600 to 800 Daltons, from 800 to 1000 Daltons, from 50 to 800 Daltons, from 50 to 600 Daltons, from 50 to 400 Daltons, from 50 to 100 Daltons, from 100 to 1000 Daltons, from 200 to 1000 Daltons, from 400 to 1000 Daltons, from 600 to 1000 Daltons, from 900 to 1000 Daltons, from 60 to 900 Daltons, or from 60 to 200 Daltons). In some examples, the cooling agent has a molecular weight of from 50 to 600 Daltons, such as from 60 to 200 Daltons.

In some examples, the cooling agent has a heat capacity of water of from −20 kJ/kg to −500 kJ/kg and a molecular weight of from 50 to 1000 Daltons, from 50 to 600 Daltons, or from 60 to 200 Daltons.

The cooling agent can be provided in any suitable form. In some examples, the cooling agent is encapsulated within the superabsorbent material at a molecular level (e.g., the cooling agent is not present as particles). For example, in various implementations the cooling agent is encapsulated within the superabsorbent material at a molecular level such that the cooling agent is dissolved and mixed with monomers at the molecular level before the polymerization starts, so that the cooling agent will be uniformly distributed or encapsulated within the polymer matrix of the superabsorbent material after the polymerization process ends. In such implementations, the cooling agent will not participate in the polymerization process so that its cooling and other functions can be preserved. In other examples, the cooling agent can be present in an aggregate form.

### Example Absorbent Composite Materials

In various implementations, the absorbent composite material comprises a cooling agent, such as one or more of the cooling agents described herein, physically encapsulated within a superabsorbent material, such as one or more of the superabsorbent materials described herein. In some examples, the absorbent composite material comprises the cooling agent in an amount of 1% or more by weight, based on the total dry weight of the absorbent composite material (e.g., 2% or more, 3% or more, 4% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 25% or more, 30% or more, 35% or more, or 40% or more). In some examples, the absorbent composite material comprises the cooling agent in an amount of 50% or less by weight, based on the total dry weight of the absorbent composite material (e.g., 45% or less, 40% or less, 35% or less, 30% or less, 25% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less). The amount of cooling agent in the absorbent composite material can range from any of the minimum values described above to any of the maximum values described above. For example, the absorbent composite material can comprise the cooling agent in an amount of from 1% to 50% by weight, based on the total dry weight of the absorbent composite material, (e.g., from 1% to 25%, from 25% to 50%, from 1% to 10%, from 10% to 20%, from 20% to 30%, from 30% to 40%, from 40% to 50%, from 1% to 40%, from 1% to 30%, from 1% to 20%, from 1% to 5%, from 5% to 50%, from 10% to 50%, from 20% to 50%, from 30% to 50%, from 2% to 40%, or from 5% to 30%). In some examples, the absorbent composite material can comprise the cooling agent in an amount of from 10% to 20% by weight, based on the total dry weight of the absorbent composite material.

The amount of cooling agent in the absorbent composite material can be selected in view of a variety of factors, such as the cooling benefits, the strength of the absorbent composite material, or a combination thereof.

In some examples, the absorbent composite material comprises the cooling agent in an amount of from 1% to 50% by weight, based on the total dry weight of the absorbent composite material, and the cooling agent comprises xylitol, sorbitol, mannitol, erythritol, urea, or a combination thereof. In some examples, the absorbent composite material comprises the cooling agent in an amount of from 1% to 50% by weight, based on the total dry weight of the absorbent composite material, and the cooling agent comprises xylitol, urea, or a combination thereof. In some examples, the absorbent composite material comprises the cooling agent in an amount of from 1% to 50% by weight, based on the total dry weight of the absorbent composite material, and the cooling agent has a heat capacity of water of from −20 kJ/kg to −500 kJ/kg. In some examples, the absorbent composite material comprises the cooling agent in an amount of from 1% to 50% by weight, based on the total dry weight of the absorbent composite material, and the cooling agent has a molecular weight of from 50 to 1000 Daltons, from 50 to 600 Daltons, or from 60 to 200 Daltons. In some examples, the absorbent composite material comprises the cooling agent in an amount of from 1% to 50% by weight, based on the total dry weight of the absorbent composite material, the cooling agent has a heat capacity of water of from −20 kJ/kg to −500 kJ/kg, and the cooling agent has a molecular weight of from 50 to 1000 Daltons, from 50 to 600 Daltons, or from 60 to 200 Daltons.

In some examples, the absorbent composite material comprises the cooling agent in an amount of from 10% to 20% by weight, based on the total dry weight of the absorbent composite material, and the cooling agent comprises xylitol, sorbitol, mannitol, erythritol, urea, or a combination thereof. In some examples, the absorbent composite material comprises the cooling agent in an amount of from 10% to 20% by weight, based on the total dry weight of the absorbent composite material, and the cooling agent comprises xylitol, urea, or a combination thereof. In some examples, the absorbent composite material comprises the cooling agent in an amount of from 10% to 20% by weight, based on the total dry weight of the absorbent composite material, and the cooling agent has a heat capacity of water of from −20 kJ/kg to −500 kJ/kg. In some examples, the absorbent composite material comprises the cooling agent in an amount of from 10% to 20% by weight, based on the total dry weight of the absorbent composite material, and the cooling agent has a molecular weight of from 50 to 1000 Daltons, from 50 to 600 Daltons, or from 60 to 200 Daltons. In some examples, the absorbent composite material comprises the cooling agent in an amount of from 10% to 20% by weight, based on the total dry weight of the absorbent composite material, the cooling agent has a heat capacity of water of from −20 kJ/kg to −500 kJ/kg, and the cooling agent has a molecular weight of from 50 to 1000 Daltons, from 50 to 600 Daltons, or from 60 to 200 Daltons.

The absorbent composite material can provide improved properties, for example relative to the superabsorbent material alone (e.g., in the absence of the cooling agent) and/or relative to mixtures of the superabsorbent material and the cooling agent (e.g., wherein the cooling agent is simply mixed with the superabsorbent material instead of being encapsulated within the super absorbent material). For example, the absorbent composite material can provide improved thermal comfort, absorbency, antimicrobial control, odor control, skin health, or a combination thereof.

In some examples, the absorbent composite material has an absorbency under load. Absorbency under load is calculated by dividing the amount of liquid (e.g., aqueous solution) absorbed by the absorbent composite material after a period of time (for example, 30 minutes, or 60 minutes to reach substantially equilibrium) by the weight of superabsorbent material (excluding the mass of the cooling agent) under a pressure (for example, 0 psi, 0.3 psi, 0.6 psi or 0.9 psi).

In some examples, the absorbency under load of the absorbent composite material is improved (e.g., increased) relative to that of the superabsorbent material in the absence of the encapsulated cooling agent. For example, the absorbency under load of the absorbent composite material can be improved (e.g., increased) relative to the superabsorbent material alone (e.g., in the absence of the cooling agent) and/or relative to mixtures of the superabsorbent material and the cooling agent (e.g., wherein the cooling agent is simply mixed with the superabsorbent material instead of being encapsulated within the super absorbent material).

In some examples, the absorbency under load of the absorbent composite material can be increased by an amount of 5% or more relative to that of the superabsorbent material in the absence of the encapsulated cooling agent (e.g., 10% or more, 15% or more, 20% or more, 25% or more, 30% or more, or 35% or more). In some examples, the absorbency under load of the absorbent composite material can be increased by an amount of 40% or less relative to that of the superabsorbent material in the absence of the encapsulated cooling agent (e.g., 35% or less, 30% or less, 25% or less, 20% or less, 15% or less, or 10% or less). The amount that the absorbency under load of the absorbent composite material is increased relative to that of the superabsorbent material in the absence of the encapsulated cooling agent can range from any of the minimum values described above to any of the maximum values described above. For example, the absorbency under load of the absorbent composite material can be increased by an amount of from 5% to 40% relative to that of the superabsorbent material in the absence of the encapsulated cooling agent (e.g., from 5% to 20%, from 20% to 40%, from 5% to 10%, from 10% to 20%, from 20% to 30%, from 30% to 40%, from 5% to 35%, from 5% to 30%, from 5% to 25%, from 5% to 15%, from 10% to 40%, from 15% to 40%, from 25% to 40%, from 35% to 40%, from 10% to 35%, or from 10% to 25%). In some examples, the absorbency under load of the absorbent composite material can be increased by an amount of from 10% to 25%, relative to that of the superabsorbent material in the absence of the encapsulated cooling agent.

In some examples, the absorbent composite material has a centrifuge retention capacity. Centrifuge retention capacity (CRC) refers to the ability of a superabsorbent material sample to retain fluids after being saturated and submitted to centrifugation under controlled conditions. The resultant retention capacity is expressed as grams of fluids retained per gram weight of the sample. The centrifuge retention capacity of the absorbent composite material is calculated by dividing the liquid absorbed by the weight of the superabsorbent material (excluding the mass of the cooling agent).

In some examples, the centrifuge retention capacity of the absorbent composite material is improved (e.g., increased) relative to that the superabsorbent material in the absence of the encapsulated cooling agent, for example relative to the superabsorbent material alone (e.g., in the absence of the cooling agent) and/or relative to mixtures of the superabsorbent material and the cooling agent (e.g., wherein the cooling agent is simply mixed with the superabsorbent material instead of being encapsulated within the super absorbent material).

In some examples, the centrifuge retention capacity of the absorbent composite material is improved relative to that the superabsorbent material in the absence of the encapsulated cooling agent. For example, the centrifuge retention capacity of the absorbent composite material can be improved (e.g., increased) by 5% or more relative to that of the superabsorbent material in the absence of the encapsulated cooling agent (e.g., 10% or more, 15% or more, 20% or more, 25% or more, 30% or more, or 35% or more). In some examples, the centrifuge retention capacity of the absorbent composite material can be improved (e.g., increased) by an amount of 40% or less relative to that of the superabsorbent material in the absence of the encapsulated cooling agent (e.g., 35% or less, 30% or less, 25% or less, 20% or less, 15% or less, or 10% or less). The amount that the centrifuge retention capacity of the absorbent composite material is improved (e.g., increased) relative to that of the superabsorbent material in the absence of the encapsulated cooling agent can range from any of the minimum values described above to any of the maximum values described above. For example, the centrifuge retention capacity of the absorbent composite material can be improved (e.g., increased) by an amount of from 5% to 40% relative to that of the superabsorbent material in the absence of the encapsulated cooling agent (e.g., from 5% to 20%, from 20% to 40%, from 5% to 10%, from 10% to 20%, from 20% to 30%, from 30% to 40%, from 5% to 35%, from 5% to 30%, from 5% to 25%, from 5% to 15%, from 10% to 40%, from 15% to 40%, from 25% to 40%, from 35% to 40%, from 10% to 35%, or from 10% to 25%). In some examples, the centrifuge retention capacity of the absorbent composite material can be improved (e.g., increased) by an amount of from 10% to 25%, relative to that of the superabsorbent material in the absence of the encapsulated cooling agent.

In some examples, the absorbency under load and the centrifuge retention capacity of the absorbent composite material can be improved (e.g., increased) by an amount of from 5% to 40% relative to that of the superabsorbent material in the absence of the encapsulated cooling agent. In some examples, the absorbency under load and the centrifuge retention capacity of the absorbent composite material can be improved (e.g., increased) by an amount of from 10% to 25%, relative to that of the superabsorbent material in the absence of the encapsulated cooling agent.

In some examples, upon liquid insult with a liquid at 35° C., the absorbent composite material exhibits an increase in temperature and the increase in temperature of the absorbent composite material is less than the increase in temperature of the superabsorbent material in the absence of the encapsulated cooling agent, for example relative to the superabsorbent material alone (e.g., in the absence of the cooling agent) and/or relative to mixtures of the superabsorbent material and the cooling agent (e.g., wherein the cooling agent is simply mixed with the superabsorbent material instead of being encapsulated within the super absorbent material). For example, the increase in temperature of the absorbent composite material can be less than the increase in temperature of the superabsorbent material in the absence of the encapsulated cooling agent by an amount of 0.1° C. or more (e.g., 0.2° C. or more, 0.3° C. or more, 0.4° C. or more, 0.5° C. or more, 0.6° C. or more, 0.7° C. or more, 0.8° C. or more, 0.9° C. or more, 1° C. or more, 1.25° C. or more, 1.5° C. or more, 1.75° C. or more, 2° C. or more, 2.25° C. or more, 2.5° C. or more, 2.75° C. or more, 3° C. or more, 3.25° C. or more, 3.5° C. or more, 3.75° C. or more, 4° C. or more, 4.25° C. or more, or 4.5° C. or more). In some examples, the increase in temperature of the absorbent composite material can be less than the increase in temperature of the superabsorbent material in the absence of the encapsulated cooling agent by an amount of 5° C. or less (e.g., 4.75° C. or less, 4.5° C. or less, 4.25° C. or less, 4° C. or less, 3.75° C. or less, 3.5° C. or less, 3.25° C. or less, 3° C. or less, 2.75° C. or less, 2.5° C. or less, 2.25° C. or less, 2° C. or less, 1.75° C. or less, 1.5° C. or less, 1.25° C. or less, 1° C. or less, 0.9° C. or less, 0.8° C. or less, 0.7° C. or less, 0.6° C. or less, 0.5° C. or less, 0.4° C. or less, or 0.3° C. or less). The amount that the amount of increase in temperature of the absorbent composite material is less than the increase in temperature of the superabsorbent material in the absence of the encapsulated cooling agent can range from any of the minimum values described above to any of the maximum values described above. For example, the increase in temperature of the absorbent composite material can be less than the increase in temperature of the superabsorbent material in the absence of the encapsulated cooling agent by an amount of from 0.1° C. to 5° C. (e.g., from 0.1° C. to 2.5° C., from 2.5° C. to 5° C., from 0.1° C. to 1° C., from 1° C. to 2° C., from 2° C. to 3° C., from 3° C. to 4° C., from 4° C. to 5° C., from 0.1° C. to 4° C., from 0.1° C. to 3° C., from 0.1° C. to 2° C., from 0.5° C. to 5° C., from 1° C. to 5° C., from 2° C. to 5° C., from 3° C. to 5° C., from 0.2° C. to 4.5° C., from 0.3° C. to 4° C., from 0.4° C. to 2° C., or from 0.4° C. to 1° C.). In some examples, the increase in temperature of the absorbent composite material can be less than the increase in temperature of the superabsorbent material in the absence of the encapsulated cooling agent by an amount of from 0.4° C. to 2° C., such as from 0.4° C. to 1° C.

In some examples, the absorbent composite material is subjected to liquid insult with a liquid at 35° C. and subsequently left at a temperature of 35° C. and a relative humidity (e.g., 70% relative humidity, 95% relative humidity) for an amount of time (e.g., 8 hours) to probe the moisture retention properties of the absorbent composite material in comparison to the superabsorbent material in the absence of the encapsulated cooling agent. In some examples, when the absorbent composite material is subjected to liquid insult with a liquid at 35° C. and subsequently left at a temperature of 35° C. and a relative humidity of 70% or more for 8 hours, then the absorbent composite material releases moisture vapor in an amount, and the amount of vapor released from the absorbent composite material is less than that of the superabsorbent material in the absence of the encapsulated cooling agent, for example relative to the superabsorbent material alone (e.g., in the absence of the cooling agent) and/or relative to mixtures of the superabsorbent material and the cooling agent (e.g., wherein the cooling agent is simply mixed with the superabsorbent material instead of being encapsulated within the super absorbent material). For example, the amount of moisture vapor released from the absorbent composite material is less than that of the superabsorbent material in the absence of the encapsulated cooling agent by an amount of 1% or more (e.g., 2% or more, 3% or more, 4% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 25% or more, 30% or more, or 35% or more). In some examples, the amount of moisture vapor released from the absorbent composite material is less than that of the superabsorbent material in the absence of the encapsulated cooling agent by an amount of 40% or less (e.g., 35% or less, 30% or less, 25% or less, 20% or less, 19% or less, 18% or less, 17% or less, 16% or less, 15% or less, 14% or less, 13% or less, 12% or less, 11% or less, 10% or less, 9% or less, 8% or less, 7% or less, 6% or less, or 5% or less). The amount by which the amount of moisture vapor released from the absorbent composite material is less than that of the superabsorbent material in the absence of the encapsulated cooling agent can range from any of the minimum values described above to any of the maximum values described above. For example, the amount of moisture vapor released from the absorbent composite material is less than that of the superabsorbent material in the absence of the encapsulated cooling agent by an amount of from 1% to 40% (e.g., from 1% to 20%, from 20% to 40%, from 1% to 10%, from 10% to 20%, from 20% to 30%, from 30% to 40%, from 1% to 35%, from 1% to 30%, from 1% to 25%, from 1% to 20%, from 1% to 5%, from 5% to 40%, from 10% to 40%, from 25% to 40%, from 5% to 35%, or from 10% to 30%). In some examples, the amount of moisture vapor released from the absorbent composite material is less than that of the superabsorbent material in the absence of the encapsulated cooling agent by an amount of from 10% to 30%.

### Absorbent Articles

Also disclosed herein are absorbent articles comprising any of the absorbent composite materials described herein. In some examples, the absorbent article can comprise a health/medical absorbent article, a household/industrial absorbent article, a personal care absorbent article, a sports/construction absorbent article, or a combination thereof. In some examples, the absorbent article is configured to be worn by a person. In some examples, the absorbent article is a diaper, toilet training pant, incontinence product, or feminine hygiene product.

In some examples, the absorbent article is configured to be subjected to a liquid insult. By way of example the liquid insult can comprise a bodily fluid. “Bodily fluid”, as used herein, refers to a fluid composition obtained from or located within a human or animal subject. Bodily fluids include, but are not limited to, sweat, urine, whole blood, blood plasma, serum, tears, semen, saliva, sputum, exhaled breath, nasal secretions, pharyngeal exudates, bronchoalveolar lavage, tracheal aspirations, interstitial fluid, lymph fluid, meningeal fluid, amniotic fluid, glandular fluid, feces, perspiration, mucous, vaginal or urethral secretion, menses, cerebrospinal fluid, and transdermal exudate. Bodily fluid also includes experimentally separated fractions of all of the preceding solutions, as well as mixtures containing homogenized solid material, such as feces, tissues, and biopsy samples. In some examples, the fluid sample comprises a bodily fluid, such as urine, feces, perspiration (e.g., sweat), mucous, vaginal or urethral secretion, or a combination thereof. In some examples, the liquid insult comprises a bodily fluid, such as urine, perspiration (e.g., sweat), menses, or a combination thereof.

In some examples, the absorbent article comprises an outer cover and an absorbent core comprising the absorbent composite material. The outer cover can, for example, have an interior surface and an exterior surface, and the absorbent core has a first surface positioned adjacent to the interior surface of the outer cover and a second surface that faces a wearer, the second surface being opposite of the first surface. In some examples, the outer cover can be liquid impermeable. The absorbent core is suitably compressible, conformable, and capable of absorbing and retaining liquid body exudates released by the wearer.

In some examples, the absorbent core further comprises one or more additional materials. For example, the absorbent core can further include a matrix of absorbent fibers, and more suitably cellulosic fluff, such as wood pulp fluff. One suitable pulp fluff is identified with the trade designation CR 1654, commercially available from Bowater, Inc. of Greenville, S.C., U.S.A. As an alternative to wood pulp fluff, synthetic fibers, polymeric fibers, meltblown fibers, short cut homofil bicomponent synthetic fibers, or other natural fibers may be used. In some examples, the absorbent core can additionally include materials such as surfactants, perfumes, odor control additives, adhesives, antimicrobial agents, and the like, and combinations thereof. In addition, the absorbent core can include foam. In some examples, the absorbent core further comprises cellulose fibers, an adhesive, or a combination thereof.

In some examples, the absorbent core comprises a nonwoven, such as meltblown, coform, spunbond, spunbond-meltblown-spunbond (SMS), bonded-carded-web (BCW), woven fabric; a perforated film; a foam layer: HYDROKNIT (a nonwoven obtainable from Kimberly-Clark Worldwide, Inc.), airlaid, cotton web and the like. In some examples, the absorbent core is airlaid.

### Methods of Making Absorbent Articles

Also disclosed herein are methods of making any of the absorbent articles disclosed herein. The absorbent articles can be formed using methods known in the art. For example, the methods can comprise depositing the absorbent composite material to form the absorbent article. In some examples, the methods can comprise making the absorbent core comprising the absorbent composite material.

While not being limited to a specific method of manufacture, the absorbent core can utilize forming drum systems, such as those described, for example, in U.S. Pat. No. 4,666,647 to Enloe et al., U.S. Pat. No. 4,761,258 to Enloe, U.S. Pat. No. 6,630,088 to Venturino et al., and U.S. Pat. No. 6,330,735 to Hahn et al., each of which is incorporated herein by reference in a manner that is consistent therewith. Examples of techniques which can introduce superabsorbent particles into a forming chamber are described in U.S. Pat. No. 4,927,582 to Bryson and U.S. Pat. No. 6,416,697 to Venturino et al., each of which is incorporated herein by reference in a manner that is consistent herewith.

In some examples, the absorbent core includes cellulose fiber and/or synthetic fiber, such as meltblown fiber, for example. Thus, in some examples, a meltblown process can be utilized, such as to form the absorbent core in a coform line. In some examples, the absorbent core can have a significant amount of stretchability. For example, the absorbent core can comprise a matrix of fibers which includes an operative amount of elastomeric polymer fibers. Other methods known in the art can include attaching superabsorbent polymer particles to a stretchable film, utilizing a nonwoven substrate having cuts or slits in its structure, and the like.

In some examples, the method comprises air laying, foam forming, or adhesive forming the absorbent composite material. In certain examples, the method comprises air laying the absorbent composite material.

In some examples, the absorbent core can be attached in an absorbent article, such as to a surge layer, an outer layer, and/or a top sheet for example, by bonding means known in the art, such as ultrasonic, pressure, adhesive, aperturing, heat, sewing thread or strand, autogenous or self-adhering, or the like, and combinations thereof. In other aspects, the absorbent core may be free-floating within the absorbent article.

In some examples, the absorbent composite material exhibits improved processability relative to the superabsorbent material and the cooling agent separately. For example, various cooling agents may have tacky or viscid surface properties. According to various implementations herein, encapsulation of the cooling agent with superabsorbent material can improve the processability of the absorbent composite material by covering the tacky or viscid surface of the cooling agent with superabsorbent material (e.g., a superabsorbent material having surface properties that are comparably less tacky or viscid).

Also disclosed herein are methods of making the absorbent composite materials. The methods can, for example, comprise forming the superabsorbent material in the presence of the cooling agent, thereby encapsulating the cooling agent within the superabsorbent material. For example, the methods can comprise polymerizing one or more monomers, optionally in the presence of a crosslinked, in the presence of the cooling agent.

A number of implementations of the disclosure have been described. Nevertheless, it will be understood that various modifications may be made without departing from the spirit and scope of the present disclosure. Accordingly, other implementations are within the scope of the following claims.

The examples below are intended to further illustrate certain aspects of the systems and methods described herein and are not intended to limit the scope of the claims.

### EXAMPLES

The following examples are set forth below to illustrate the methods and results according to the disclosed subject matter. These examples are not intended to be inclusive of all aspects of the subject matter disclosed herein, but rather to illustrate representative methods and results. These examples are not intended to exclude equivalents and variations of the present disclosure, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of measurement conditions, e.g., component concentrations, temperatures, pressures and other measurement ranges and conditions that can be used to optimize the described process.

### Example 1—Cooling Agents Encapsulated with Superabsorbent Material to Suppress Heating and Water Vaporization

Thermal comfort has been identified to be one of the top benefits to deliver overall comfort for absorbent articles. Several strategies have been used to address thermal comfort with some successes, though more effective methods and materials are still highly desirable. Described herein are superabsorbent materials (“SAMs”) that can be used in absorbent garments to improve thermal comfort by reducing the heat upon swelling by aqueous media and suppressing water evaporation from swollen superabsorbent materials to manage the micro-environment of absorbent articles.

For example, the superabsorbent materials disclosed herein can comprise a crosslinked polymeric hydrogel with encapsulated cooling agents, wherein the cooling agents can comprise neutral and hydrophilic small molecules. The cooling agents can, for example, have a heat capacity of water (or an aqueous solution) of −50 kJ/kg. In some examples, the cooling agents can have a molecular weight of 600 Daltons or less. The cooling agents can also help suppress moisture release from the swollen hydrogel materials.

Examples of cooling agents include, but are not limited to, xylitol, sorbitol, mannitol, erythritol, and urea.

The superabsorbent materials disclosed herein can, for example, comprise one or more cooling agents encapsulated within the hydrogel matrix. For example, the superabsorbent materials can comprise the cooling agent in an amount of from 1% to 40% based on the total material weight.

Examples of crosslinked polymeric hydrogels include, but are not limited to, neutral and charged polymeric hydrogel materials. Examples of charged polymeric hydrogels include, for example, negatively charged, positively charged, and zwitterionic polymers. Examples of negatively charged polymeric hydrogels include, but are not limited to, polyacrylic acid-based materials, AMPS (acrylamido-2-methylpropane sulfonic acid) based hydrogel materials, and hydrogels of copolymers of AA-AMPS. Examples of positively charged hydrogel materials include, but are not limited to, polyimine and polyamine. Examples of crosslinked neutral polymeric hydrogels include, but are not limited to, PEG-based and polysaccharide-based materials.

Also disclosed herein are methods of making the hydrogel materials with encapsulated cooling agents. For example, monomer(s) and crosslinker(s) are polymerized along with the cooling agents and other reagents to form hydrogels with encapsulated cooling agents. In some examples, the hydrogels are then dried. The dried hydrogels can be processed into different forms, such as superabsorbent material particles.

Also disclosed herein are methods of use and articles of manufacture comprising the hydrogel materials with encapsulated cooling agents. For example, products, such as absorbent articles, can contain the superabsorbent materials disclosed herein. Examples of absorbent articles include, but are not limited to, diapers, incontinence garments, feminine pads and liners. Also disclosed herein are absorbent articles containing the materials for the benefit of thermal comfort, antimicrobial control, odor control, skin health, or a combination thereof.

The cooling agent can reduces the overall temperature of the absorbent system when insulted. This in turn reduces the temperature of the microclimate of the absorbent article while in use. The cooling agent can also help to suppress moisture release from the swollen hydrogel materials to the benefit of the overall capacity/retention of the absorbent system. Encapsulation of the cooling agent within the SAM particles can make the absorbent composite materials process more practically. The addition of the cooling agent can have a positive contribution to the AUL of the SAM. The absorbent composite materials can also benefit antimicrobial control, odor control, or skin health, depending on the functionality of the cooling agents.

### Example 2

Example superabsorbent materials comprising a hydrogel and a cooling agent were prepared. It was found that the cooling effect when the cooling agents were encapsulated within the hydrogel was the same as the cooling effect when the cooling agents were disposed outside the hydrogel (e.g., not encapsulated). It was surprisingly found that the cooling agents not only contributed a cooling effect upon swelling with water or bodily fluids, but also contributed to the absorbency of the SAM (e.g., Absorbency Under Load (AUL) and centrifuge retention capacity (CRC)). For hydrogel materials with encapsulated xylitol, the xylitol can further provide antimicrobial and odor control benefits.

### Experiments

Preparation of p(AA-MBAA) particles. All polymerization reactions were carried out in a 1 L reactor. The reaction temperature was controlled by a temperature controller and the reaction mixture was stirred mechanically. For a typical reaction, 252 mL of milli-Q water was cooled in the reactor to 3° C. and 63 g of NaOH was added. After the NaOH was completely dissolved, 150 g of acrylic acid (AA) was added to the reactor. The reaction mixture was cooled for 15 minutes, followed by the addition of 3 g N,N-methylenebisacrylamide (MBAA). After all solids were dissolved, the mixture was warmed to 15° C. and degassed with N₂ for 1 hour. Then, the mixture was warmed to 40° C. and 0.45 mL of N,N,N′,N′-tetramethyl ethylenediamine (TEMED) in 5 mL of milli-Q water was added, followed by the addition of 0.6 g of ammonium persulfates (APS) in 5 mL of milli-Q water. Gelling normally started about 3 minutes later. The stirring was stopped 30 minutes after gelling was completed. Then, the gel was cured at 40° C. for 3.5 hours. The gel was removed from the reactor and crushed by hand into chunks. The chunks were then dried at 70° C. for 48 hours. The dried hydrogel materials were ground into particles and sieved into a proper size range (~300-600 μm). These particles are designated as AA-MBAA (100-2) (e.g., acrylic acid and MBAA in a weight ratio of 100 to 2).

Preparation of p(AA-MBAA) with xylitol. The procedure was the same as for the p(AA-MBAA) particles, except 30 g of xylitol was added after MBAA was completely dissolved. These particles are designated as AA-MBAA-X (100-2-20).

Preparation of p(AA-MBAA) with xylitol. The procedure was the same as for the p(AA-MBAA) particles, except 15 g of xylitol was added after MBAA was completely dissolved. These particles are designated as AA-MBAA-X (100-2-10).

Preparation of p(AA-MBAA) with urea. The procedure was the same as for the p(AA-MBAA) particles, except 30 g of urea was added after MBAA was completely dissolved. These particles were designated as AA-MBAA-U (100-2-20).

Preparation of p(AA-MBAA) particles with urea. The procedure was the same as for the p(AA-MBAA) particles, except 15 g of urea was added after MBAA was completely dissolved. These particles were designated as AA-MBAA-U (100-2-10).

Preparation of p(AA-MBAA) particles with xylitol and urea. The procedure was the same as for the p(AA-MBAA) particles, except 15 g of xylitol and 15 g of urea were added after MBAA was completely dissolved. These particles were designated as AA-MBAA-X-U (100-2-10-10).

### Experimental Results

The effect of swelling ratio and cooling agents on the temperature increase of commercial superabsorbent materials was examined by exposing the superabsorbent materials (with and without cooling agents) to different volumes of 0.9% saline (e.g., different swelling ratios). Table 1 shows the results for Evonik-5630, and Table 2 shows the results for SG-200 from SDP Global. At each swelling ratio, the temperature increase was smaller when the xylitol was present.

TABLE 1
Impact of SAM swelling on temperature (Evonik).

| | | 0.9% | Initial | Highest | T | |
|---|---|---|---|---|---|---|
| E-5630 | Xylitol | Saline | T | T | Change | Time |
| (g) | (g) | (ml) | (° C.) | (° C.) | (° C.) | (min) |
| 2.5 | 0 | 10 | 23.0 | 27.1 | +4.1 | <1 |
| 2.5 | 0.5 | 10 | 23.0 | 26.3 | +3.3 | <1 |
| 2.5 | 0 | 25 | 23.6 | 25.6 | +2.0 | <1 |
| 2.5 | 0.5 | 25 | 23.8 | 25.1 | +1.3 | <1 |
| 2.5 | 0 | 50 | 25.2 | 26.5 | +1.3 | <1 |
| 2.5 | 0.5 | 50 | 25.3 | 26.2 | +0.9 | <1 |

TABLE 2
Impact of SAM swelling on temperature (SDP global).

| | | 0.9% | | Highest | | |
|---|---|---|---|---|---|---|
| SG-200 | Xylitol | Saline | Initial T | T | T Change | Time |
| (g) | (g) | (ml) | (° C.) | (° C.) | (° C.) | (min) |
| 2.5 | 0 | 10 | 23.0 | 27.1 | +4.1 | <1 |
| 2.5 | 0.5 | 10 | 23.0 | 25.8 | +2.8 | <1 |
| 2.5 | 0 | 25 | 23.0 | 25.1 | +2.1 | <1 |
| 2.5 | 0.5 | 25 | 23.2 | 24.2 | +1.0 | <1 |
| 2.5 | 0 | 50 | 23.2 | 24.2 | +1.0 | <1 |
| 2.5 | 0.5 | 50 | 23.4 | 24.0 | +0.6 | <1 |

The cooling effect of xylitol inside and outside the hydrogel materials was compared. For xylitol “inside” the hydrogel, the xylitol was encapsulated within the hydrogel as described above. For xylitol “outside” the hydrogel, the hydrogel was prepared and then subsequently mixed with xylitol. A control sample of just hydrogel with no xylitol was also tested. The total mass was kept constant across all tests (SAM+xylitol, or just SAM). The results are summarized in Table 3. The temperature increase upon swelling was smaller when the xylitol was present. Surprisingly, the cooling effect of xylitol was the same for xylitol encapsulated within the hydrogel or xylitol outside the hydrogel.

TABLE 3
Cooling Impact of Xylitol inside or outside of SAM.

| | | | 0.9% | Initial | Highest | T |
|---|---|---|---|---|---|---|
| | SAM | Xylitol | saline | T | T | Change |
| | (g) | (g) | (ml) | (° C.) | (° C.) | (° C.) |
| PAA-MBAA | 2.08 | 0.42 | 25 | 23.4 | 24.7 | +1.3 |
| (100-2) | | | | | | |
| PAA-MBAA-X | 2.50 | 0.00 | 25 | 23.5 | 24.8 | +1.3 |
| (100-2-20) | | | | | | |
| PAA-MBAA | 2.27 | 0.23 | 25 | 23.4 | 25.2 | +1.8 |
| (100-2) | | | | | | |
| PAA-MBAA-X | 2.50 | 0.00 | 25 | 23.4 | 25.2 | +1.8 |
| (100-2-10) | | | | | | |
| PAA-MBAA | 2.50 | 0.00 | 25 | 23.4 | 25.6 | +2.2 |
| (100-2) | | | | | | |

The cooling effect of urea inside and outside the hydrogel materials was compared. For urea “inside” the hydrogel, the urea was encapsulated within the hydrogel as described above. For urea “outside” the hydrogel, the hydrogel was prepared and then subsequently mixed with urea. A control sample of just hydrogel with no urea was also tested. The total mass was kept constant across all tests (SAM+urea, or just SAM). The results are summarized in Table 4. The temperature increase upon swelling was smaller when the urea was present. Surprisingly, the cooling effect of urea was substantially the same for urea encapsulated within the hydrogel or urea outside the hydrogel.

TABLE 4
Cooling Impact of Urea inside or outside of SAM.

| | | | 0.9% | Initial | Highest | T |
|---|---|---|---|---|---|---|
| | SAM | Urea | saline | T | T | Change |
| | (g) | (g) | (ml) | (° C.) | (C) | (° C.) |
| PAA-MBAA | 2.08 | 0.42 | 25 | 22.6 | 23.8 | +1.2 |
| (100-2) | | | | | | |
| PAA-MBAA-U | 2.50 | 0.00 | 25 | 22.3 | 23.6 | +1.3 |
| (100-2-20) | | | | | | |
| PAA-MBAA | 2.27 | 0.23 | 25 | 22.6 | 24.3 | +1.7 |
| (100-2) | | | | | | |
| PAA-MBAA-U | 2.50 | 0.00 | 25 | 22.8 | 24.3 | +1.5 |
| (100-2-10) | | | | | | |
| PAA-MBAA | 2.50 | 0.00 | 25 | 23.4 | 25.6 | +2.2 |
| (100-2) | | | | | | |

The cooling effect of the combination of xylitol and urea was investigated. The xylitol and urea were encapsulated within the hydrogel as described above. The results are summarized in Table 5. The cooling effect of xylitol and urea was additive.

TABLE 5
Cooling Impact of Xylitol & Urea inside or outside of SAM

| | | 0.9% | Initial | Highest | T |
|---|---|---|---|---|---|
| | SAM | saline | T | T | Change |
| | (g) | (ml) | (° C.) | (° C.) | (° C.) |
| PAA-MBAA-X-U | 2.50 | 25 | 22.7 | 24.1 | +1.4 |
| (100-2-10-10) | | | | | |
| PAA-MBAA-X-U | 2.50 | 50 | 22.8 | 23.4 | +0.6 |
| (100-2-10-10) | | | | | |

The absorbency of hydrogel materials with and without xylitol was compared. Table 6 compares the Absorbency Under Load (AUL) of superabsorbent materials with and without xylitol, as well as the effect of xylitol inside and outside the superabsorbent materials.

More specifically, Row A of Table 6 shows the AUL of an example where xylitol was mixed with (e.g., outside) the superabsorbent material, while Row B shows the AUL of an example where xylitol was physically encapsulated within (e.g., inside) the superabsorbent material. Surprisingly, xylitol encapsulated within the superabsorbent material has a positive contribution to the AUL in comparison with xylitol outside the superabsorbent material. More specifically, the AUL at 0.5 hours increased by 5.3%, the AUL at 1 hour increased by 9.9%, and the AUL at 4 hours increased by 9.2% for the material with xylitol encapsulated within the superabsorbent material (Row B) in comparison with xylitol outside (e.g., mixed with) the superabsorbent material (Row A).

Row C of Table 6 shows the AUL for an example material including only AA and MBAA, with no xylitol. Row D shows the AUL for a similar material including xylitol, the xylitol being physically encapsulated within the SAM, where the amount of AA in row D is decreased relative to row C by an amount approximately equivalent to the amount of xylitol added in Row D, such that the total mass of the SAM is the approximately same between Rows C and D. As can be seen from Table 6, the percent decrease in the amount of AA in Row D relative to Row C is 17.6%. If xylitol did not contribute to absorbency, then the AUL of Row D would be expected to similarly decrease by ~17.6%. However, the AUL at 1 hour decreased by only 10.3%, and the AUL at 4 hours decreased by only 8.9%. As such, the decrease in the AUL was surprisingly lower than expected for the SAM with xylitol physically encapsulated therein relative to the SAM in the absence of the xylitol, indicating that the physical encapsulation of xylitol within the SAM unexpectedly has a positive contribution to the absorbency.

TABLE 6
Impact of Xylitol on AUL (0.3 psi in 0.9% saline, average from duplicates)

| | | SAM | Xylitol | SAM + X | 0.5 hours | 1.0 hours | 4.0 hours |
|---|---|---|---|---|---|---|---|
| A | PAA-MBAA (100-2) | 0.1672 | 0.0344 | 0.2016 | 18.2 | 18.3 | 18.7 |
| B | PAA-MBAA-X | 0.2018 | 0 | 0.2018 | 19.2 | 20.2 | 20.5 |
| | (100-2-20) | | | | | | |
| | | SAM | AA | X | MBAA | 1.0 hours | 4.0 hours |
| C | PAA-MBAA | 0.2000 | 0.1961 | 0 | 0.0039 | 22.4 | 22.3 |
| | (100-2) | | | | | | |
| D | PAA-MBAA-X | 0.2006 | 0.1644 | 0.0328 | 0.0040 | 20.2 | 20.4 |
| | (100-2-20) | | | | | | |

The absorbency of hydrogel materials with and without urea were also compared. Table 7 compares the Absorbency Under Load (AUL) of superabsorbent materials with and without urea, as well as the effect of urea inside and outside the superabsorbent materials.

More specifically, Row A of Table 7 shows the AUL of an example where urea was mixed with (e.g., outside) the superabsorbent material, while Row B shows the AUL of an example where urea was physically encapsulated within (e.g., inside) the superabsorbent material. Surprisingly, urea encapsulated within the superabsorbent material has a positive contribution to the AUL in comparison with urea outside the superabsorbent material. More specifically, the AUL at 0.5 hours increased by 2.7%, the AUL at 1 hour increased by 5.2%, and the AUL at 4 hours increased by 4.2% for the material with urea encapsulated within the superabsorbent material (Row B) in comparison with urea outside (e.g., mixed with) the superabsorbent material (Row A).

Row C of Table 7 shows the AUL for an example material including only AA and MBAA, with no urea. Row D shows the AUL for a similar material including urea, the urea being physically encapsulated within the SAM, where the amount of AA in row D is decreased relative to row C by an amount approximately equivalent to the amount of urea added in Row D, such that the total mass of the SAM is the approximately same between Rows C and D. Similar to the results for xylitol, the decrease in the AUL was surprisingly lower than expected for the SAM with urea physically encapsulated therein relative to the SAM in the absence of the urea.

TABLE 7
Impact of urea on AUL (0.3 psi in 0.9% saline, average from duplicates)

| | | SAM | Urea | SAM + U | 0.5 hours | 1.0 hours | 4.0 hours |
|---|---|---|---|---|---|---|---|
| A | PAA-MBAA (100-2) | 0.1658 | 0.0344 | 0.2002 | 18.1 | 18.6 | 18.6 |
| B | PAA-MBAA-U (100-2-20) | 0.2009 | 0 | 0.2009 | 18.6 | 19.6 | 19.4 |

| | | SAM | 0.5 hours | 1.0 hours | 4.0 hours |
|---|---|---|---|---|---|
| C | PAA-MBAA (100-2) | 0.2007 | 21.8 | 22.2 | 22.3 |
| D | PAA-MBAA-U (100-2-20) | 0.2021 | 19.1 | 19.4 | 19.6 |

The dynamic vapor release profiles for hydrogels with and without urea or xylitol were compared.

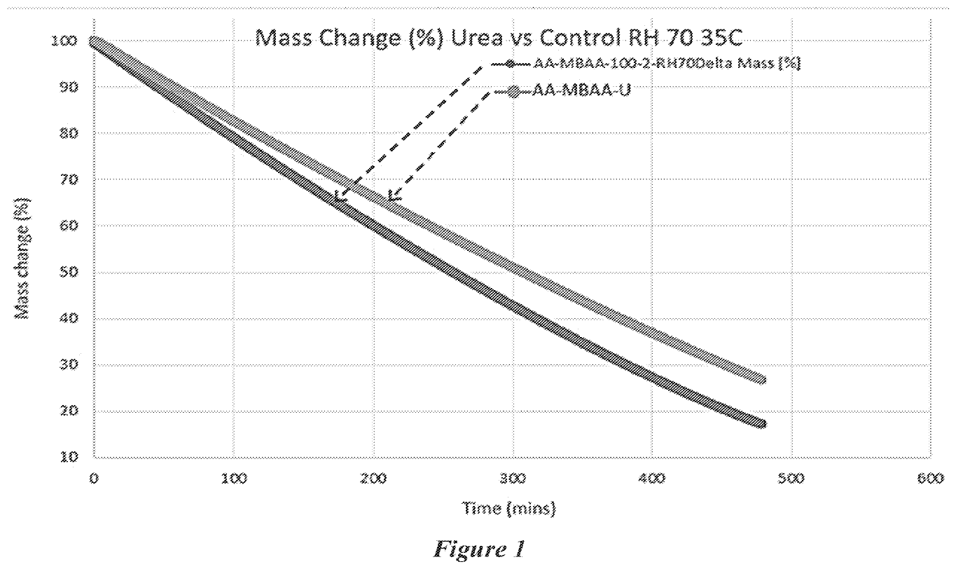

FIG. 1 compares the water loss profiles from swollen SAMs at 35° C. under 70% relative humidity (RH). The swollen SAMs with encapsulated urea release water moisture slower than the SAMs without urea.

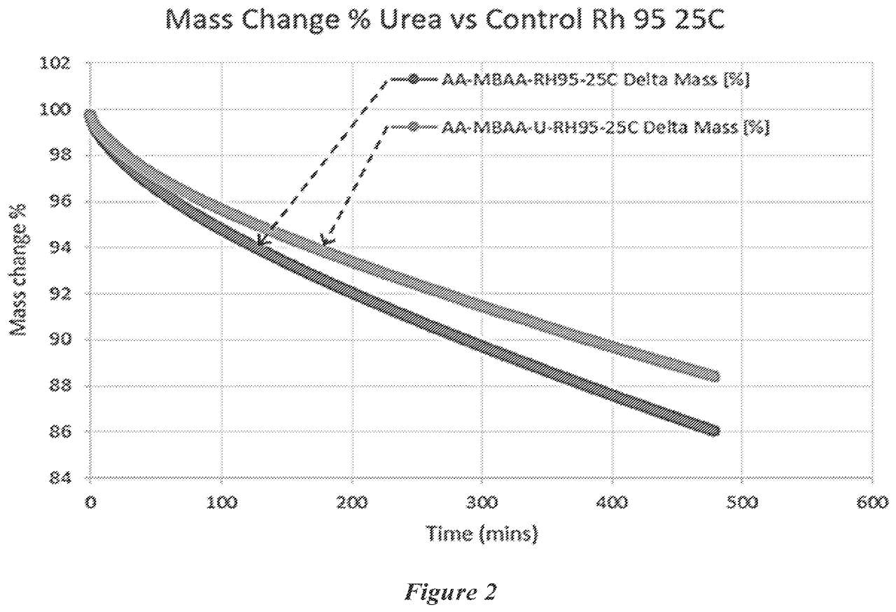

FIG. 2 compares the water loss profiles from swollen SAMs at 25° C. under 95% relative humidity (RH). The swollen SAMs with encapsulated urea release water moisture slower than the SAMs without urea.

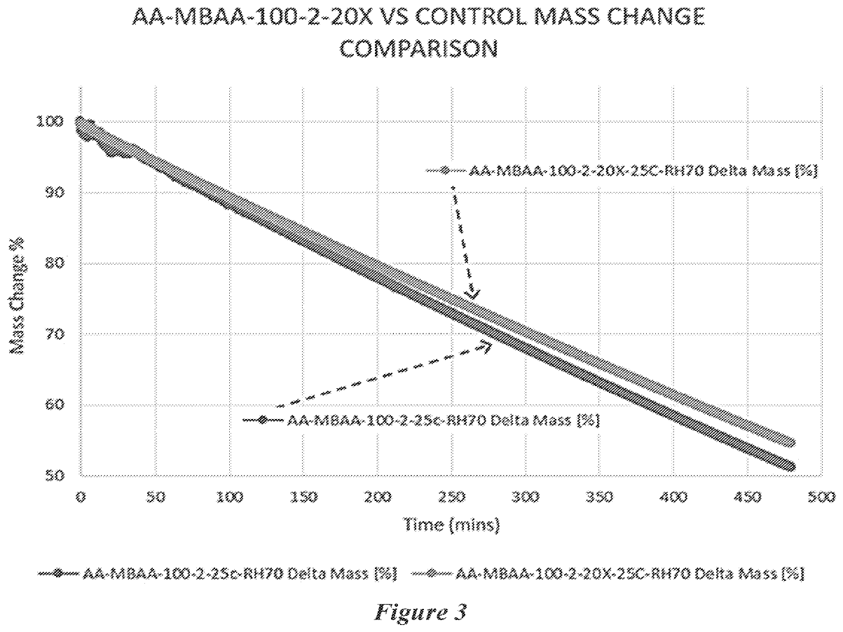

FIG. 3 compares the water loss profiles from swollen SAMs at 25° C. under 70% relative humidity (RH). The swollen SAMs with encapsulated xylitol released water moisture slower than the SAMs without xylitol. In various implementations, it is desirable that the absorbent composite with a cooling agent can exhibit slower moisture release rates after liquid insults so that it can help to reduce microclimate moisture content in an absorbent garment such as diaper or pants during actual usage conditions. The quantification of the moisture release (rates & capacity) was conducted by employing a dynamic vapor sorption system from Surface Measurement Systems LTD. NA (DVS Adventure) by measuring the moisture release speed and amount with the desired amount of composite samples (e.g., 5-100 milligrams typically) under desired temperature (e.g. 25° C. or 35° C., etc.) and relative humidity (e.g. 70% or 95%, etc.).

The storage modulus and absorbency properties of superabsorbent materials as a function of xylitol loading was investigated.

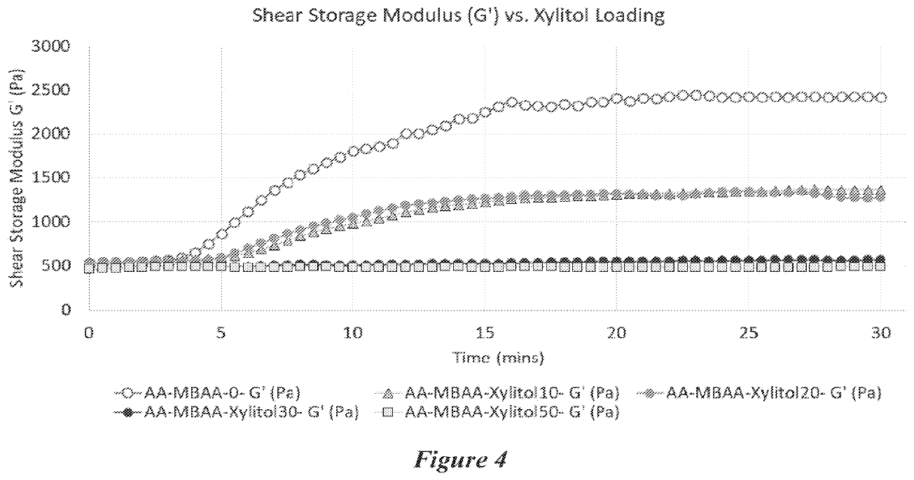

FIG. 4 shows the shear storage modulus as a function of xylitol loading. Although higher cooling agent loading levels can maximize the cooling benefits, excessive cooling agent loading can lead to lower gel strength as shown in FIG. 4. By employing a dynamic gel strength measurement system, ElastoSens Bio*, from Rheolution Inc., it was found that higher than 20% loading levels of a cooling agent can lead to significant loss of gel strength, as indicated by almost flat shear storage modulus G′(Pa) during gel swelling in FIG. 4 for samples with higher than 20% loading levels of a cooling agent.

Table 8 summarizes the absorbency properties as a function of xylitol loading.

More specifically, the amount of xylitol physically encapsulated within the SAM is 0% in Row A, 9% in Row B, 16% in Row C, 23% in Row D, and 33% in Row E of Table 8.

Comparing the AUL at 1 hour in Table 8, the AUL decreased by 5% for row B relative to Row A (e.g., when the amount of xylitol increased by 9%), 10% for row C relative to Row A (e.g., when the amount of xylitol increased by 16%), 20.7% for row D relative to Row A (e.g., when the amount of xylitol increased by 23%), and 29.7% for Row E relative to row A (e.g., when the amount of xylitol increased by 33%).

Comparing the AUL at 4 hours in Table 8, the AUL decreased by 3.7% for row B relative to row A (e.g., when the amount of xylitol increased by 9%), 8.4% for row C relative to Row A (e.g., when the amount of xylitol increased by 16%), 19.7% for row D relative to Row A (e.g., when the amount of xylitol increased by 23%), and 29.3% for Row E relative to row A (e.g., when the amount of xylitol increased by 33%).

Comparing the CRC in Table 8, the CRC decreased by 8.6% for row B relative to row A (e.g., when the amount of xylitol increased by 9%), 9% for row C relative to Row A (e.g., when the amount of xylitol increased by 16%), 19.5% for row D relative to Row A (e.g., when the amount of xylitol increased by 23%), and 24.3% for Row E relative to row A (e.g., when the amount of xylitol increased by 33%).

As such, in general the decrease in the AUL and CRC was surprisingly lower than expected for the SAM with xylitol physically encapsulated (Row B-Row E) therein relative to the SAM in the absence of the xylitol (Row A).

TABLE 8
Absorbency properties vs. xylitol loading.

| | | AUL | AUL | AUL | | Vortex |
|---|---|---|---|---|---|---|
| | AA-MBAA-X | 0.5 hr | 1.0 hr | 4.0 hrs | CRC | time (s) |
| A | 100-2-0 (0%) | | 22.4 | 22.3 | 23.1 | 90 |
| B | 100-2-10 (9%) | 21.1 | 21.3 | 21.5 | 21.2 | 94 |
| C | 100-2-20 (16%) | 19.7 | 20.2 | 20.5 | 21.1 | 92 |
| D | 100-2-30 (23%) | 18.1 | 18.2 | 18.3 | 19 | 108 |
| E | 100-2-50 (33%) | 16.2 | 16.6 | 16.6 | 18.1 | 114 |

AUL at 0.9% saline
0.3 psi
CRC (1 hr) in 0.9% saline
Vortex time in 0.9% saline

### EXEMPLARY ASPECTS

In view of the described compositions, devices, systems, and methods, herein below are described certain more particularly described aspects of the inventions. The particularly recited aspects should not, however, be interpreted to have any limiting effect on any different claims containing different or more general teachings described herein or that the “particular” aspects are somehow limited in some way other than the inherent meanings of the language and formulas literally used therein.

Example 1: An absorbent composite material comprising: a superabsorbent material; and a cooling agent; wherein the cooling agent is physically encapsulated within the superabsorbent material.

Example 2: The absorbent composite material of any examples herein, particularly example 1, wherein the cooling agent is not chemically reacted with the superabsorbent material.

Example 3: The absorbent composite material of any examples herein, particularly example 1 or example 2, wherein the superabsorbent material comprises a plurality of particles, such that the absorbent composite material comprises the cooling agent encapsulated within the plurality of particles.

Example 4: The absorbent composite material of any examples herein, particularly example 3, wherein the plurality of particles have an average particle size of 20 to 2000 micrometers (microns, μm), such as from 20 to 1000 micrometers, from 60 to 500 micrometers, or from 300 to 600 micrometers.

Example 5: The absorbent composite material of any examples herein, particularly examples 1-4, wherein the superabsorbent material comprises a polymer, such as a crosslinked polymer.

Example 6: The absorbent composite material of any examples herein, particularly examples 1-5, wherein the superabsorbent material comprises a polymeric hydrogel, such as a crosslinked polymeric hydrogel.

Example 7: The absorbent composite material of any examples herein, particularly examples 1-6, wherein the superabsorbent material is derived from acrylic acid, an acrylamide, or a combination thereof.

Example 8: The absorbent composite material of any examples herein, particularly examples 1-7, wherein the superabsorbent material comprises a polyacrylic acid-based hydrogel material, an AMPS (acrylamido-2-methylpropane sulfonic acid) based hydrogel material, a polyimine-based hydrogel material, a polyamine-based hydrogel material, a PEG-based hydrogel material, a polysaccharide-based hydrogel material, or a combination thereof.

Example 9: The absorbent composite material of any examples herein, particularly examples 1-8, wherein the superabsorbent material is derived from acrylic acid, N,N-methylenebisacrylamide (MBAA), or a combination thereof.

Example 10: The absorbent composite material of any examples herein, particularly examples 1-9, wherein the cooling agent has a heat capacity of water of from −20 kJ/kg to −500 kJ/kg.

Example 11: The absorbent composite material of any examples herein, particularly examples 1-10, wherein the cooling agent has a molecular weight of from 50 to 1000 Daltons, such as from 50 to 600 Daltons.

Example 12: The absorbent composite material of any examples herein, particularly examples 1-11, wherein the cooling agent comprises an anhydrous salt (e.g., a sugar alcohol, urea), a hydrous salt (e.g., a salt hydrate), or a combination thereof.

Example 13: The absorbent composite material of any examples herein, particularly examples 1-12, wherein the cooling agent comprises a sugar alcohol, such as xylitol, sorbitol, mannitol, erythritol, or a combination thereof.

Example 14: The absorbent composite material of any examples herein, particularly examples 1-13, wherein the cooling agent comprises xylitol, sorbitol, mannitol, erythritol, urea, or a combination thereof.

Example 15: The absorbent composite material of any examples herein, particularly examples 1-14, wherein the cooling agent comprises xylitol, urea, or a combination thereof.

Example 16: The absorbent composite material of any examples herein, particularly examples 1-15, wherein the absorbent composite material comprises the cooling agent in an amount of from 1% to 50% by weight, based on the total dry weight of the absorbent composite material, such as from 10% to 20%.

Example 17: The absorbent composite material of any examples herein, particularly examples 1-16, wherein the absorbent composite material has an absorbency under load and the absorbency under load of the absorbent composite material is improved (e.g., increased) relative to that of the superabsorbent material in the absence of the encapsulated cooling agent.

Example 18: The absorbent composite material of any examples herein, particularly example 17, wherein the absorbency under load of the absorbent composite material is increased by an amount of from 5% to 40%, such as from 10% to 25%, relative to that of the superabsorbent material in the absence of the encapsulated cooling agent.

Example 19: The absorbent composite material of any examples herein, particularly examples 1-18, wherein the absorbent composite material has a centrifuge retention capacity and the centrifuge retention capacity of the absorbent composite material is improved (e.g., increased) relative to that the superabsorbent material in the absence of the encapsulated cooling agent.

Example 20: The absorbent composite material of any examples herein, particularly example 19, wherein the centrifuge retention capacity of the absorbent composite material is improved by an amount of from 5% to 40%, such as from 10 to 25%, relative to that of the superabsorbent material in the absence of the encapsulated cooling agent.

Example 21: The absorbent composite material of any examples herein, particularly examples 1-20, wherein, upon liquid insult with a liquid at 35° C., the absorbent composite material exhibits an increase in temperature and the increase in temperature of the absorbent composite material is less than the increase in temperature of the superabsorbent material in the absence of the encapsulated cooling agent.

Example 22: The absorbent composite material of any examples herein, particularly example 21, wherein the increase in temperature of the absorbent composite material is less than the increase in temperature of the superabsorbent material in the absence of the encapsulated cooling agent by an amount of from 0.1° C. to 5° C., such as from 0.4° C. to 2° C., or from 0.4° C. to 1° C.

Example 23: The absorbent composite material of any examples herein, particularly examples 1-22, wherein, when the absorbent composite material is subjected to liquid insult with a liquid at 35° C. and subsequently left at a temperature of 35° C. and 70% relative humidity or more for 8 hours, then the absorbent composite material releases moisture vapor in an amount, and the amount of vapor released from the absorbent composite material is less than that of the superabsorbent material in the absence of the encapsulated cooling agent.

Example 24: The absorbent composite material of any examples herein, particularly example 23, wherein the amount of moisture vapor released from the absorbent composite material is less than that of the superabsorbent material in the absence of the encapsulated cooling agent by an amount of from 1% to 40%, such as from 10% to 30%.

Example 25: The absorbent composite material of any examples herein, particularly examples 1-24, wherein the absorbent composite material provides improved thermal comfort, absorbency, antimicrobial control, odor control, skin health, or a combination thereof relative to the superabsorbent material in the absence of the encapsulated cooling agent.

Example 26: An absorbent article comprising the absorbent composite material of any examples herein, particularly examples 1-25.

Example 27: The absorbent article of any examples herein, particularly example 26, wherein the absorbent article is configured to be worn by a person.

Example 28: The absorbent article of any examples herein, particularly example 26 or example 27, wherein the absorbent article is a diaper, toilet training pant, incontinence product, or feminine hygiene product.

Example 29: The absorbent article of any examples herein, particularly examples 26-28, wherein the absorbent article is configured to be subjected to a liquid insult.

Example 30: The absorbent article of any examples herein, particularly example 29, wherein the liquid insult comprises contact with urine, menses, perspiration, or a combination thereof.

Example 31: The absorbent article of any examples herein, particularly examples 26-30, wherein the absorbent article comprises an outer cover and an absorbent core comprising the absorbent composite material.

Example 32: The absorbent article of any examples herein, particularly example 31, wherein the outer cover has an interior surface and an exterior surface, and the absorbent core has a first surface positioned adjacent to the interior surface of the outer cover and a second surface that faces a wearer, the second surface being opposite of the first surface.

Example 33: The absorbent article of any examples herein, particularly example 31 or example 32, wherein the absorbent core is airlaid.

Example 34: The absorbent article of any examples herein, particularly examples 31-33, wherein the absorbent core further comprises one or more additional materials, such as cellulose fibers, an adhesive, or a combination thereof.

Example 35: A method of making the absorbent article of any examples herein, particularly examples 26-34, wherein the method comprises depositing the absorbent composite material to form the absorbent article.

Example 36: The method of any examples herein, particularly example 35, wherein the method comprises air laying the absorbent composite material.

Example 37: The method of any examples herein, particularly example 35 or example 36, wherein the absorbent composite material exhibits improved processability relative to the superabsorbent material and the cooling agent separately.

Example 38: The method of any examples herein, particularly examples 35-37, wherein the method further comprises making the absorbent composite material.

Example 39: A method of making the absorbent composite material of any examples herein, particularly examples 1-25.

Other advantages which are obvious and which are inherent to the various implementations described herein will be evident to one skilled in the art. It will be understood that certain features and sub-combinations are of utility and may be employed without reference to other features and sub-combinations. This is contemplated by and is within the scope of the claims. Since many possible implementations may be made of the present disclosure without departing from the scope thereof, it is to be understood that all matter herein set forth or shown in the accompanying drawings is to be interpreted as illustrative and not in a limiting sense.

The methods of the appended claims are not limited in scope by the specific methods described herein, which are intended as illustrations of a few aspects of the claims and any methods that are functionally equivalent are intended to fall within the scope of the claims. Various modifications of the methods in addition to those shown and described herein are intended to fall within the scope of the appended claims. Further, while only certain representative method steps disclosed herein are specifically described, other combinations of the method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

1. An absorbent composite material comprising:
a superabsorbent material; and
a cooling agent;
wherein the cooling agent is physically encapsulated within the superabsorbent material.

2. The absorbent composite material of claim 1, wherein the cooling agent is not chemically reacted with the superabsorbent material.

3. The absorbent composite material of claim 1, wherein the superabsorbent material comprises a plurality of particles, such that the absorbent composite material comprises the cooling agent encapsulated within the plurality of particles.

4. The absorbent composite material of claim 3, wherein the plurality of particles have an average particle size of 20 to 2000 micrometers (microns, μm).

5. The absorbent composite material of claim 1, wherein the superabsorbent material comprises a polymer.

6. The absorbent composite material of claim 1, wherein the superabsorbent material comprises a polymeric hydrogel.

7. The absorbent composite material of claim 1, wherein the superabsorbent material is derived from acrylic acid, an acrylamide, or a combination thereof.

8. The absorbent composite material of claim 1, wherein the superabsorbent material comprises a polyacrylic acid-based hydrogel material, an AMPS (acrylamido-2-methylpropane sulfonic acid) based hydrogel material, a polyimine-based hydrogel material, a polyamine-based hydrogel material, a PEG-based hydrogel material, a polysaccharide-based hydrogel material, or a combination thereof.

9. The absorbent composite material of claim 1, wherein the superabsorbent material is derived from acrylic acid, N,N-methylenebisacrylamide (MBAA), or a combination thereof.

10. The absorbent composite material of claim 1, wherein the cooling agent has a heat capacity of water of from −20 kJ/kg to −500 kJ/kg.

11. The absorbent composite material of claim 1, wherein the cooling agent has a molecular weight of from 50 to 1000 Daltons.

12. The absorbent composite material of claim 1, wherein the cooling agent comprises an anhydrous salt, a hydrous salt, or a combination thereof.

13. The absorbent composite material of claim 1, wherein the cooling agent comprises a sugar alcohol.

14. The absorbent composite material of claim 1, wherein the cooling agent comprises xylitol, sorbitol, mannitol, erythritol, urea, or a combination thereof.

15. (canceled)

16. The absorbent composite material of claim 1, wherein the absorbent composite material comprises the cooling agent in an amount of from 1% to 50% by weight, based on the total dry weight of the absorbent composite material.

17. The absorbent composite material of claim 1, wherein:
the absorbent composite material has an absorbency under load and the absorbency under load of the absorbent composite material is improved relative to that of the superabsorbent material in the absence of the encapsulated cooling agent;
the absorbent composite material has a centrifuge retention capacity and the centrifuge retention capacity of the absorbent composite material is improved relative to that the superabsorbent material in the absence of the encapsulated cooling agent;
upon liquid insult with a liquid at 35° C., the absorbent composite material exhibits an increase in temperature and the increase in temperature of the absorbent composite material is less than the increase in temperature of the superabsorbent material in the absence of the encapsulated cooling agent;
when the absorbent composite material is subjected to liquid insult with a liquid at 35° C. and subsequently left at a temperature of 35° C. and 70% relative humidity or more for 8 hours, then the absorbent composite material releases moisture vapor in an amount, and the amount of vapor released from the absorbent composite material is less than that of the superabsorbent material in the absence of the encapsulated cooling agent;
the absorbent composite material provides improved thermal comfort, absorbency, antimicrobial control, odor control, skin health, or a combination thereof relative to the superabsorbent material in the absence of the encapsulated cooling agent;
or a combination thereof.

18.-25. (canceled)

26. An absorbent article comprising the absorbent composite material of claim 1.

27. (canceled)

28. The absorbent article of claim 26, wherein the absorbent article is a diaper, toilet training pant, incontinence product, or feminine hygiene product.

29.-34. (canceled)

35. A method of making the absorbent article of claim 26, wherein the method comprises depositing the absorbent composite material to form the absorbent article.

36. (canceled)

37. (canceled)

38. (canceled)

39. A method of making the absorbent composite material of claim 1.
